(19) United States
(12) Patent Application Publication
(10) Pub. No.: US 2009/0326336 A1
(43) Pub. Date: Dec. 31, 2009

(54) Process for comprehensive surgical assist system by means of a therapy imaging and model management system (TIMMS)

(76) Inventors: Heinz Ulrich Lemke, Kuessaberg (DE); Leonard F. Berliner, Staten Island, NY (US)

Correspondence Address: Louis A. Piccone, 519 Kirchner Road, Dalton, MA, 01226, US

(21) Appl. No.: 12/213,979

(22) Filed: Jun. 25, 2008

(51) Int. Cl. A61B 5/00; A61B 19/00; G06N 5/02; G06Q 50/00

(52) U.S. Cl. 600/300; 128/898; 706/52; 705/3

(57) ABSTRACT

This invention provides a process and system for a comprehensive surgical assist system, called a Therapy Imaging and Model Management System (TIMMS), which combines and integrates all of the necessary information and communication technology; workflow analysis, data processing and data synthesis; interactive interfaces between surgeon and mechatronic devices; and, cognitive agents; to provide comprehensive assistance and guidance throughout complex medical and surgical therapies, such as image guided surgery. The components of this invention, which are modular, scalable and may be distributed in location, act synergistically to provide functionality and utility that exceeds the sum of its individual parts.

A method of performing surgery on a patient comprising the step of comparing a chosen patient's data to statistical data in a repository of patient data to develop a patient specific model, wherein the data comprises information from two or more sub databases selected from the group consisting of workflow data, electronic medical records, diagnostic data, biological data, measurement data, anatomical data, physiological data, genetic data, molecular data, imaging data, chemical data, clinical laboratory data, simulated data, coordinate data and surgical result and wherein the patient specific model aids in the preoperative, operative or post operative phase of surgery performed in real time on the patient.

### CROSS-REFERENCES TO RELATED APPLICATIONS

This application is related to provisional application No. [To Be Added] in the name of Heinz Lemke and Leonard F. Berliner from which priority is claimed.

### FIELD OF THE INVENTION

This invention relates to a process and system for a comprehensive surgical assist system, which combines and integrates all of the necessary information and communication technology; workflow analysis, data processing and data synthesis; interactive interfaces between surgeon and mechatronic devices; and, cognitive agents; to provide comprehensive assistance and guidance throughout complex medical and surgical therapies, such as image guided surgery.

### BACKGROUND OF THE INVENTION

It has been predicted that there will be an increase in the demand of from 14% to 47% for all surgical services by the year 2020. Difficulties which are already now apparent in the operating room, such as the lack of seamless integration of surgical assist systems into the surgical workflow, will be amplified in the near future. There are many rudimentary surgical assist systems in development or which are employed in the operating room, mostly in an isolated fashion which does not allow for the comprehensive use of information from a wide variety of sources. Their routine use in the operating room, however, is impeded by the absence of appropriate integration technology and standards.

Appropriate use of information and communication technology and mechatronic systems as part of a re-engineered medical and surgical workflow would be a useful contribution to solve the problems. However, up until now the appropriate information technology infrastructure, as well as communication and interface standards (which allow data interchange between surgical system components in the operating room) have not been integrated into a single, consistent functioning process.

A variety of problems and limitations of image guided surgery have been inadequately addressed in the prior art. These barriers to, and limitations of, effective surgical workflow and operating room infrastructure include:
1. Inefficient, ineffective and redundant processes;
2. Inflexible systems of operation;
3. Ergonomic deficiencies which hinder the workflow;
4. Inadequate and incomplete presentation of intraoperative and perioperative data (text and images);
5. Lack of methods for the real-time presentation of “soft knowledge” (such as, background medical information; alternative surgical actions and strategy, in case of unanticipated events in the operating room);
6. Absent or inefficient processes for scheduling and tracking of patients, personnel, operating rooms, and equipment;
7. Inefficient processes for effectively integrating image-guided and mechatronic surgery processes into the actual surgical workflow;
8. Lack of consistent or standardized working practices, guidelines, or workflows;
9. Inflexibility of surgical workflows to adapt to specific patient responses that occur throughout a surgical procedure;
10. Absence of standardized interfacing of surgical devices and systems;
11. Lack of quantified information on workflow and error handling;
12. Inadequate communication across disciplines (such as radiology and surgery).

As a result, the true benefits of image guided surgery to individual patients and to society cannot be fully obtained with the isolated tools that are currently available. While numerous devices are commercially available for image guided surgery, the absence of a cohesive process which integrates these tools into the workflow severely limits the functionality of those tools. Therefore, the overall success of image guided surgery is currently limited.

Consequently, image guided surgery, without the appropriate integration into a comprehensive process, often adds cost to health care, without providing the anticipated benefits to patients and society that is expected of today's advancements in technology. The current invention provides a process for the successful and practical development of the modem operating room which provides, to the surgeon, an environment that can improve surgical outcomes and help ensure patient safety.

### SUMMARY OF THE INVENTION

The present invention is directed to a method of performing surgery on a patient comprising the step of comparing a chosen patient's data to statistical data in a repository of patient data to develop a patient specific model, wherein the data comprises information from two or more sub databases selected from the group consisting of workflow data, electronic medical records, diagnostic data, biological data, measurement data, anatomical data, physiological data, genetic data, molecular data, imaging data, chemical data, clinical laboratory data, simulated data, coordinate data and surgical result and wherein the patient specific model aids in the preoperative, operative or post operative phase of surgery performed in real time on the patient.

The present invention is also directed to a repository, communications and computer service oriented architecture for surgical assistance comprising: a first element consisting of two or more electronic repositories selected from the group consisting of workflow data, electronic medical records, diagnostic data, anatomical data, physiological data, genetic data, molecular data, imaging data, chemical data, clinical laboratory data, biological data, simulated data, measurement data, coordinate data and surgical result; a second element consisting of a means for communications between medical personnel, the two or more electronic repositories and one or more engines making up a third element; and, a third element consisting of one or more engines which generate, analyze, evaluate, or manage input and output; and wherein, the three elements are connected to each other such that patient specific data input or manipulated by a medical professional can be compared to statistical data or generic models, said statistical data or generic models derived from previously entered patient specific data.

The present invention is still yet further directed to an electronic patient specific model comprising: a computer based data set comprising a first set of information specific to one patient, a second set of information from a statistical sampling of other patents having similar symptoms or diagnosis and a third set of information comprising an analysis of the first two sets of information and wherein the third set of information is useful in the diagnosis or treatment of a patient.

### DESCRIPTION OF THE DRAWINGS

FIG. 1 shows a typical schematic of a network of hardware components in accordance with the present invention with the following items identified: (120) Enterprise-Wide Electronic Medical Record including PACS, HIS, RIS, Data Warehouse; (125) TIMMS Medical Workstation; (130) TIMMS Surgical Workstation; (135) Servers for Engines and Repositories; (135A) Server Containing TIMMS Repositories; (135B) Server Containing TIMMS Engines; (140) Interfaces for Operative Tools; (140A) Mechatronic and other Surgical Devices; (140B) Positioning Devices; (140C) Monitoring and Sensor Devices; (140D) Model Building Devices; (140E) Imaging Devices e.g. X-ray, CT, MR, US; (140F) Navigation Devices; (145) Control and Input Devices; (150) Security Devices; (155) Monitors and Visualization Devices; (210A) Imaging and Biosensors Engine; (210B) Modeling Engine; (210C) Simulation Engine; (210D) Kernel for Workflow and Knowledge and Decision Management Engine; (210E) Representation and Visualization Engine; (210F) Intervention Engine; (210G) Validation Engine; (215A) Images and Signals Repository; (215B) Modeling Tools Repository; (215C) Computing Tools Repository; (215D) Workflow and Knowledge and Decision Tools Repository; (215E) Representation Tools Repository; (215F) Devices and Mechatronic Tools Repository; (215G) Validation Tools Repository; (220) Repository for Models (Simulated Objects); (225) Repository for Workflows, Evidence-based Medicine, Cases; (230) Information and Communication Technology Infrastructure; (235) Data and Information; (240) Models and Intervention Records.

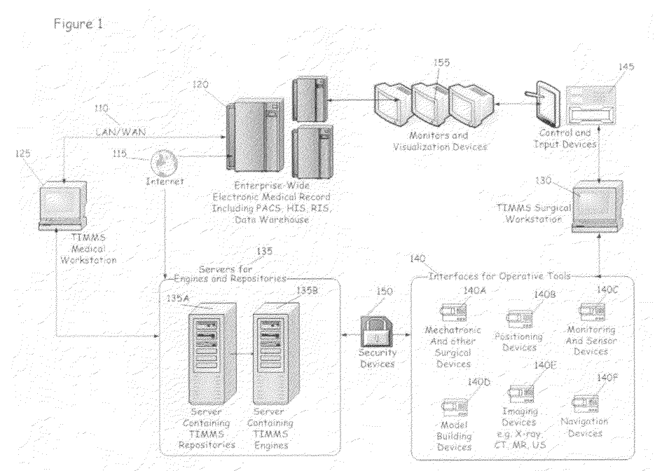

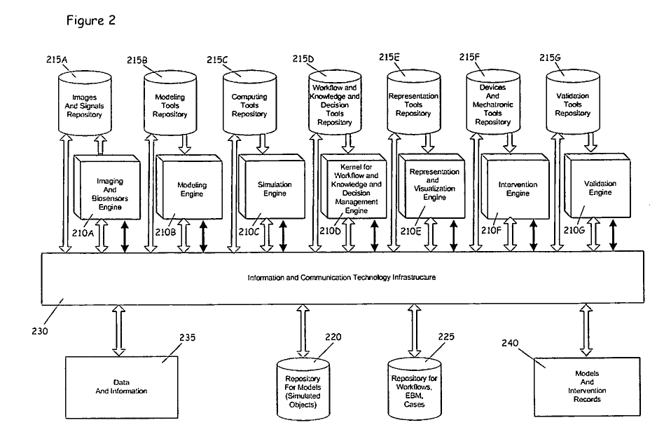

The TIMMS network described in FIG. 1 may extend over standard LAN/WAN (110) and/or Internet devices (115), to facilitate input of data into the TIMMS system from an Enterprise-Wide Electronic Medical Record (120) (which may include PACS Systems, standard Hospital Information Systems, Radiology Information Systems, and Data Warehouses).

A typical TIMMS Medical Workstation (125), used for pre-operative and post-operative planning and analysis, can consist of a CPU, input devices, one or more monitors, connection to the TIMMS network, and the TIMMS software.

The TIMMS Surgical Workstation (130), used for intra-operative functions, can comprise a CPU, input devices, one or more monitors, connection to the TIMMS network, and the TIMMS software. There can be interfaces with the proprietary navigational and mechatronic devices used during the specific surgical procedure as well as input and output interfaces to patient monitoring and imaging devices.

One or more servers, linked to the TIMMS network, can contain sufficient memory for the storage of data repositories required for the surgical intervention, Server for TIMMS repository (135A). There can be sufficient functionality to perform the tasks as specified by the various engines, Server for TIMMS engines (135B).

A TIMMS system provides Interfaces for Operative Tools (140) which can consist of an Interface Distribution Device and Gateway. This is a mechanical/electronic device which provides for the interconnectivity between the various TIMMS hardware, software, and network components. It contains computing ability to direct the flow of information/data between the required locations.

Interfaces are provided for such tools as: 1) Mechatronic and other Surgical Devices (140A); 2) Positioning Devices (140B); 3) Monitoring and Sensor Devices (140C); 4) Model Building Devices (140D); 5) Imaging Devices (such as X-ray, CT, MR, US) (140E); 6) Navigation Devices (140F). Whenever possible, industry standard computer and telecommunication interfaces are employed between components, such as DICOM and HL7, for example. The TIMMS system also accommodates proprietary computer and telecommunication connections and interfaces.

Standard computer control and input devices, such as computer mouse, keyboard, joystick, controller pad, may be employed for input of information, data, and commands.

A variety of interfaces for Human-computer interaction (HCI) may be employed such as Human Language Interfaces for voice recognition and language translation for verbal input for all appropriate computer controlled functions, voice recognition could be used for computer commands as well as input of data, and, language translation capabilities would be useful for remote telemedicine, or whenever a participant would benefit from real-time language translation.

Security Devices (150), such as USB security keys, or other hardware or software devices or dongles, that contain licenses for multiple products, or multiple and separate functions within one product, may be used to allow authorized use of TIMMS or external components.

With appropriate security and authorization, external devices (such as mechatronic or navigation systems) will be able pass their data streams and instructions through the TIMMS network. At the same time, information could be passed in a precise manner between the TIMMS and the external system in a manner that would enhance the success of the overall process.

Monitors and Visualization Devices or displays (155) may be utilized, depending upon the specific application.

The displays may require capabilities for displaying text; medical images; physiologic data; stereoscopic images; as well as other capabilities.

Functionality for interactivity may be provided with data input and control through standard devices such as computer mouse, touch screen, as well as voice activated input.

The Physician workstation may only require conventional CRT and/or flat panel monitors. For specific application, interactive and/or stereoscopic capable monitors may be required. (Glasses assisted or glasses free monitors may be employed.)

The monitors employed for the TIMMS Surgical Workstation (130) may employ any or all of the following: single monitor; an array of monitors; a widescreen wall-mounted monitor; head-mounted viewing devices; stereoscopic monitors or head-mounted stereoscopic viewers; interactive monitors; as well as other viewing devices.

FIG. 2 is a schematic of one embodiment of a TIMMS architecture, demonstrating the functional relationships between various engines; repositories; information and communication technology infrastructure; and outside sources of data and information, and sites of data storage. The open arrows refer to data exchange, and the solid arrows indicate control.

The overall system is modular and scalable depending upon the specific needs of the end-user, and the components may be distributed among sites within a hospital environment, or other medical facility at which Surgical, Interventional Radiology, or other therapeutic procedures are planned, performed, analyzed, and/or monitored. The system is sufficiently robust for the most simple to the most complex surgical procedures. Any and every surgical procedure can benefit from a TIMMS Implementation.

The software aspects of the present invention can function within a hardware environment, or TIMMS network, as shown in FIG. 2, consisting of clusters of computer workstations in multiple environments; a high-speed network which can be DICOM compliant; and, computer devices and interfaces for storage, retrieval, routing, analysis, processing, and transmission of a variety of a variety of medical images and data, including patient-specific physiological data and images; and, is composed of a plurality of interconnected software components, or TIMMS software.

### DETAILED DESCRIPTION OF THE INVENTION

For purposes of the present invention the following terms are defined below:

“TIMMS” is an acronym and common law trademark standing for Therapy Imaging and Model Management System.

“MGT” is an acronym standing for Model Guided Therapy.

“PSM” is an acronym standing for Patient Specific Model.

“IGT” is an acronym standing for Image Guided Therapy.

“EMR” is an acronym standing for Electronic Medical Record.

“MBE” is an acronym standing for Model Based Evidence.

“EBM” is an acronym standing for Evidence Based Medicine.

The term “data” means human perceptible elements of electronic information (i.e., text or graphics) which are gathered, associated, created, formatted, edited, prepared, or otherwise processed in forming a unified collection of such information storable as a distinct entity

“Model” is something (as a similar object or a construct) used to help visualize or explore something else (as the living human body) that cannot be directly observed or experimented on. Models can similarly be defined as one or more simulated objects.

For purposes of the present invention, the term “process” is a coherent sequence of steps undertaken by a program to manipulate data such as an internal or external data-transfer operation, handling an interrupt, or evaluation of a function.

“Processing” is a method for, or, an apparatus performing systematic operations upon data or information exemplified by functions such as data or information transferring, merging, sorting, and computing (e.g., arithmetic operations or logical operations).

The term “Medical Images” refers to medical and radiological images such as pictures, x-rays, CT scans, MRI's, echocardiograms, full body scans, angiogram images, etc. to name just a few. A medical image is a representation of a part of the body in one or more types of media, such as paper, film, CRT, flatscreen, drawing, etc.

The “adaptive workflow tool” is a cognitive subprogram of the TIMMS workflow tool which makes revisions to the reference workflow thereby forming an executing workflow.

“Cognitive agents” are herein defined as software modules, containing some form of intelligence, which, with some degree of autonomy and adaptability, carry out functions or tasks.

“Engine” may be defined as a software module which can be executed on an appropriate computing machine or central processing unit.

“Image-Centric World View”, as an approach to medical imaging, is defined as a view of patient care which is limited to the realm of the medical images themselves.

“Model-Centric World View”, as employed by TIMMS, is defined as a view of patient care which extends beyond the realm of the medical images, and includes a wider variety of information, relating to the patient, which, when integrated with the images, provides a more comprehensive and robust view of the patient.

“Mechatronics” is defined as the synergistic combination of mechanical engineering, electronic engineering, and software engineering.

“Repository” may be defined as an integrated hardware and software structure which has the capability of storing and making available, data and/or data processing tools.

“TIMMS Service Oriented Architecture” or SOA service components are categorized into engines (functional and process components), repositories and interaction components.

TIMMS SOA service components (i.e. repositories, functional and process components as well as the TIMMS infrastructure component) may include or share a uniform interaction component.

An “Information Object Definition” or IOD is a software or digital representation of a real object (e.g., CT Image, Study, etc.). An “Information Object” is a list of characteristics (Attributes) which completely describe the object and which can be recognized by software. The formal description of an Information Object generally includes a description of its purpose and the attributes it possesses.

A “Service Class”, a group of operations that a user might want to perform on particular Information Objects. Formally, a structured description of a service which is supported by cooperating DICOM Application Entities using specific DICOM Commands acting on a specific class of Information Object.

Service-Object Pair (SOP): The combination of a DICOM Information Object and the Service Class which operates upon that object.

A “tool” is defined according to its regular English definition and includes a software program which can perform one or more tasks. For example, the EMR tool can automatically retrieve and display all pertinent medical records. The imaging tool can automatically retrieve and display all pertinent medical images. The surgical modeler tool can provide an integrated environment between all available imaging and the patient specific model. The treatment outcomes predictor tool can reference material and statistical processes from one or more local or networked repositories. The complication tool can evaluate all input for possible or actual complications of the surgical procedures. The update tool can update the specific patients EMR and patient model with all relevant test data generated during the surgical procedure. Graphing tool can graph and track one or more test result such as for example, blood gases, heart beat, respirations, etc. The network tool can access medical databases from other physicians, practices, hospitals and regions for accumulating data available from those databases into the TIMMS system. The adaptive workflow tool updates the executing workflow as the procedure progresses. For example if a rib turns out to be in the planned needle path, new trajectories and coordinates are calculated and provided as output.

A “treatment simulation” is a plan generated and then reviewed and modified by the surgeon.

The “patient safety agent” will automate patient safety processes during the pre-procedural patient assessment and monitoring stage of patient treatment.

Statistical patient data is data from two or more patient's that has been analyzed and assigned a statistical value such as, for example, mean, average, top half, bottom half, top 1 percentile, or any statistical or medical sub-classification thereof based on a particular criteria. For example, mean of the subclass of patients suffering from a particular complication.

“Workflow data” concerns information on the steps needed to engage in a process. Workflow information regarding a particular surgery may be broad or detailed and may include individual steps which may or may not be taken as well as any sub-steps which can be performed. For example, a surgical procedure can be defined as broad workflow information such as 1) patient is prepped and brought into operating room, 2) operation is performed and 3) patient is transported to recovery room. The workflow may also be extremely detailed for example, an incision is made in the patients skin, if an artery is cut then proceed to repair said artery or if no artery is cut, proceed to clean out infected area using sterile solution.

“Diagnostic data” refers to data with can be used to make a diagnosis and includes the diagnosis itself.

“Biological data” concerns any biological information regarding a patients' body.

“Measurement data” is measurement information from a patients' body.

“Anatomical data” refers to information concerning a patient's anatomy.

“Physiological data” concerning information regarding the physiology of a patients' body.

“Pathological data” is data generated by a pathologist or concerning pathology data available from a patient or his/her tissues.

“Genetic data” refers to any information which may be available from a patients' genome and includes sequencing information, interpretation of sequencing information and genetic screening data for example.

“Molecular data”, is data concerning the molecular structure of one or more structures in a patients' body.

“Imaging data” is information available from one or more medical images.

“Chemical data” is data concerning any chemical test which can be performed in the body substantially overlapping clinical laboratory data, but that which may be immediately available to the physician without having another person participating. For example, oxygenation level of the blood is a chemical test performed on a patient, but which can be performed by a monitor on a patient's finger as opposed to clinical laboratory data which must be preformed in a lab.

“Clinical laboratory data” is data based on standard clinical laboratory data and includes that information available from tests such a bloods tests, urinalysis, pap smears, biopsy data, pathology reports, etc. which typically must be performed by a clinical laboratory.

“Simulated data” is data based on a statistical data.

“Coordinate data” refers to a set of three dimensional coordinate system covering all or a portion of a patient's body so that each location of a patients body has a three dimensional coordinate. For example, a tumor in a patients head can be mapped and assigned coordinates with respect to the rest of the patient's body. The physician, or a surgeon in particular, can then develop a treatment plan based on the size of the tumor, the proximity to other structures in the patients body assigned coordinates, and then guide surgery, at a remote location, or using automated surgical instruments.

A “surgical procedure” as defined here, may include all facets of pre-operative, intra-operative and post-operative assessment, planning and patient care.

“Surgical result” is data concerning patient outcome and can include the myriad of information encompassed by the term patient outcome, including mortality, morbidity, cost, days in hospital, medication usage, etc.

As one of ordinary skill in the art will appreciate each of the following sets of data may have substantial overlap with one or more of the other sets of data: workflow data electronic medical records, diagnostic data, biological data, measurement data, anatomical data, physiological data, genetic data, molecular data, imaging data, chemical data, clinical laboratory data, simulated data, coordinate data and surgical result. For purposes of the present invention, when two or more of these sets of data are used together, the individual data will be different so that the information is not duplicative.

The present invention is directed to informative and useful compilations of electronic data concerning a given patient and a statistical sampling of other patients. The present invention is also directed to a method of using the informative and useful compilations of electronic data before, during or after surgery. In particular the present invention is directed to an electronic patient specific model (ie, generically an informative and useful compilation of electronic data) comprising: a computer based data set comprising a first set of information specific to one patient, a second set of information from a statistical sampling of other patents having similar symptoms or diagnosis and a third set of information comprising an analysis of the first two sets of information and wherein the third set of information is useful in the diagnosis or treatment of a patient.

The present invention provides a process and system for a comprehensive surgical assist system, which combines and integrates all of the necessary information and communication technology; workflow analysis, data processing and data synthesis; interactive interfaces between surgeon and mechatronic devices; and, cognitive agents; to provide comprehensive assistance and guidance throughout complex medical and surgical therapies, such as image guided surgery. The components of this invention, which are modular, scalable and may be distributed in location, act synergistically to provide functionality and utility that exceeds the sum of its individual parts.

The skilled artisan will appreciate that the electronic patient specific model may be a novel combination of elements that are known in the art which are available in real time to a physician, in particular a surgeon, treating a patient.

The electronic patient specific model is a computer based data set comprising a first set of information specific to one patient, a second set of information from a statistical sampling of other patents having similar symptoms or diagnosis and a third set of information comprising an analysis of the first two sets of information and wherein the third set of information is useful in the diagnosis or treatment of a patient.

The patient specific model puts all known data concerning a patient in one location and provides the surgeon or other medical professional with real time comprehensive information regarding the patient, and regarding a number of other patients having a similar diagnosis, surgery or condition to improve the patient's medical outcome. For example, the patient specific model would provide, in real time, relevant data, as data points regarding the specific patient. The patient specific model would also provide statistical information regarding a number of other patients having a similar condition. The patient specific model would similarly provide a analysis of the two sets of information to provide the physician with useful information.

By way of further example, take a patient presenting with a brain tumor which is close in proximity to a major blood vessel. The tumor may be neutralized using a number of different therapies or surgeries. The specific patients tumor is mapped and assigned coordinates sufficient to describe the dimensions and location of the tumor. The blood vessel is then similarly mapped and assigned coordinates. All appropriate tests are performed on the patient. A statistical analysis is done to compare the specific patient data to one or more statistical functions on similar data regarding all input patients having the same condition. The function may be average, mean, high low range, etc. The analysis can then break this statistical result down by outcome according to the particular therapy used on past patients to determine that workflow path which has the best statistical patient outcome to compare therapy alternatives. For example, surgical intervention may be ruled out in favor of chemotherapy because of the complication rate of damaging the nearby blood vessel. The workflow can then be modified to suit the physician's skills, the hospitals resources, risk of complications, etc. The surgeon has great flexibility to modify the workflow to fit the patients' best probable outcome in view of the totality of the circumstance. The coordinate data can be used to plot growth rate of the tumor compared to statistical averages, or means for example, to determine best timing of medical intervention. At the time of surgery, the coordinates of the tumor can then be used to guide the 3 dimensional radiofrequency destruction of the tumor while the surgeon is at a remote location. Afterwards the specific patient's data is input into the system as a part of the statistical information to aid future patients.

The skilled artisan will appreciate the current state of software development. There are already numerous computer programs or software available to perform the functions necessary to accomplish the present invention. For example there are programs which can be used to create, manage, and analyze data input into a database. There are also “off the shelf” software that will perform statistical functions, such as, mean and average, on data. There are medical specific software for managing patient data. And there is a wide range of software for any of the individual applications, or parts to be combined to create the complex combination of old elements which the subject invention can be considered to be.

There is also the expertise within the skill of an ordinary software engineer to choose, combine and integrate the currently available software to create the overall system herein described. With this in mind, the present invention will now be described in detail.

For complex surgical procedures to achieve their full potential it is necessary to develop specific strategies and processes which are incorporated into a single, multi-faceted process for the improvement of the surgical outcomes. The tool described herein, combines all of the fundamental functional components that are required to carry out the medical and surgical evaluation, management and therapy of an image and information guided surgical procedure. Technologies that have not been specifically developed for the operating room environment, such as artificial intelligence and data mining, can be combined with medical information technologies and image processing technologies, as well as electronic and mechanical devices developed specifically for the operating room.

The resulting system provides the information technology-based infrastructure necessary for surgical and interventional workflow management of the modern operation room in a manner that cannot be achieved by current technologies alone. The concept and design of the current invention is based on the assumption that significant improvement in the quality of patient care, as well as ergonomic and economic improvements in the operating room can only be achieved by means of an information technology infrastructure for data, image, information, model, and tool communication. As stated above the design of this invention takes into account modem software engineering principles, and clarifies the right position of interfaces and relevant standards for a surgical assist system in general and their components specifically.

Therapy Imaging and Model Management System (TIMMS) can provide aid, support and information throughout the entire surgical procedure, from pre-operative assessment and surgical planning, throughout the actual surgical procedure and post-operative care, and into the post-operative stages of patient evaluation and quality assurance assessment.

The Therapy Imaging and Model Management System (TIMMS) is an integral part of a complex digital infrastructure, for the planning and implementation of surgical procedures, by combining advanced adaptive expert systems and cognitive agents with all of the available imaging and surgical tools and techniques into a comprehensive system. This system then interacts, through an adaptive workflow process, with the surgeon in image and information guided surgery, and other related forms of medical management in the peri-operative period.

As conceived the capabilities of this process and system include: 1) organization of the various sources of input data prior to, during, and after the operation; 2) management of the various workflows which compose a complex surgical procedure, including the ability to learn and adapt to changes that occur in the patient, and then to adapt the workflow model accordingly; 3) technical support and guidance for the diagnostic and interventional components throughout the surgical procedure; 4) improvement of situational awareness throughout all aspects of the operating room by taking into account the special needs of imaging and modeling tools within the surgical workflow; 5) provision of information, in real-time, regarding operative and peri-operative processes; 6) the ability to respond to best practices and variances in actual patient care; 7) incorporation of standard interfaces to seamlessly integrate information technology and mechatronics systems into the operating room; 8) the ability to exchange feedback between human or mechatronic operators; and, 9) scalability and modularity

The components of this invention, which are modular, scalable and may be distributed in location, act synergistically to provide functionality and utility that exceeds the sum of its individual parts. The components include: seven “Engines”, as defined above, which work independently and dependently, and account for all facets of complex medical and surgical procedures. The seven engines are: 1) Imaging and Biosensors; 2) Modeling; 3) Simulation; 4) Kernel for Workflow and Knowledge and Decision Management; 5) Visualization and Representation; 6) Intervention; 7) Validation.

Associated Repositories may also be linked to each of the seven engines. These include: 1) images and signals Repository for the Imaging and Biosensors engine; 2) modeling tools Repository for the Modeling engine; 3) computing tools repository for the Simulation engine; 4) workflow and knowledge and decision tools repository for the kernel for workflow and knowledge and decision management engine; 5) representation tools repository for the visualization and representation engine; 6) devices and mechatronic tools repository for the intervention engine; 7) validation tools repository for the validation engine; 8) models; 9) references such as workflow models, evidence-based medical data, case-based medical data.

The system provides for real-time data mining from these repositories during the performance of the surgical procedure.

The Kernel for Workflow and Knowledge and Decision Management Engine may be composed of one or more parts or functionalities comprising for example, a central computing kernel (or “brain”) of the system may use different forms of logic, different database structuring, cognitive agents and other forms of artificial intelligence, depending on the specific applications of the procedure or procedures being performed. Cognitive agents may be defined as software modules, containing some form of intelligence, which, with some degree of autonomy and adaptability, carry out functions or tasks.

Cognitive agents may be called by the workflow engine when executing a given activity component/element of a given workflow. In general, cognitive agents are part of the Kernel for workflow and knowledge and decision management, but there may be also be part of and/or accessible to the other engines of TIMMS.

Kernel for Workflow and Knowledge and Decision information and Communication may be incorporated allowing for intercommunication and interactivity between all components of the TIMMS.

All of the engines, tools, repositories, ICT infrastructure, data sources, and the operative team are linked, through a distributed network, providing for the full functionality of TIMMS, including planning, guidance, learning, and data mining and processing.

The Information/Communication Technology infrastructure used by TIMMS includes, structures, objects, processes and interfaces from well established standardized sources, to ensure compatibility. This includes, but is not limited to: IHE (Integrating the Healthcare Enterprise); HIS (Hospital Information System); RIS (Radiology Information System); PACS (Picture Archiving and Communication System); DICOM (Digital Imaging and Communications in Medicine); and, HL7 (Health Level 7)

The present invention is also directed to an underlying construct or approach to patient management entitled a Model-Centric View. Traditionally, the approach to medical imaging when applied to clinical aspects of patient care has been limited to the realm of the images themselves. This has been called the Image-Centric World View. However, the approach to medical imaging employed by TIMMS is extended far beyond the realm of the images. In the Model-Centric World View a wide variety of information, relating to the patient, can be integrated with the images, providing a more comprehensive and robust view of the patient. TIMMS employs the Model-Centric World View, providing and utilizing all available data for surgical interventions.

The incorporation and utilization of workflow processes, within the Kernel for Workflow and Knowledge and Decision Management is central to the functioning of TIMMS. TIMMS can employ an adaptive workflow engine that is flexible and capable of learning and providing guidance throughout the procedure. A reference workflow, which provides the basic framework for a surgical procedure, evolves into an executing workflow, which is patient specific and is based on the model-centric view of the patient that also evolves throughout the entire patient encounter. For example, modifications to the executing workflow may be based on feedback from physiologic monitoring of the patient, from the surgeon, from operative robots, from operative haptic devices, from stored data within repositories.

Modifications to the executing workflow are in synchronization with updates to the Patient Model by the modeling engine. The selected surgical Reference Workflow is extracted from the appropriate repository during the planning stage of the surgical procedure.

The TIMMS system may also employ data collection which is automated for all aspects of the pre-surgical evaluation, intra-operative procedures, and post-operative evaluation. Methodology in the form of sub-programs, may be provided for the application of statistical processes to the accumulated data.

The methodology for error handling and validation is built into the system so that variations in human performance, as well as machine performance, and patient response are factored in, and learned from, at any given step of the surgical procedure. The system contains the functionality to achieve refinements in medical and surgical “best practices” and to facilitate quality improvement programs. Further illustrative examples of end use applications of the TIMMS System in prospective medical research projects which will be more easily achieved through the automated collection, monitoring and measuring of large volumes of data, with numerous variables.

TIMMS may be interfaced with outside data sources. According to the present invention interfaces are provided for the input of data and information from the outside world which are then processed and utilized by the functional components of TIMMS and stored within the repositories.

Interfaces are also provided for the output of various models, intervention records, data and information that have been synthesized within the TIMMS structure.

Interfaces are provided for the necessary hardware and software computer processes involved in surgical planning, such as information technology and communications, imaging, visualization and representation, simulation, modeling, robotics and other forms of mechatronics. This provision may be vendor-independent as long as there is conformance to industry standards. Provision may also be made for proprietary equipment through appropriate interfaces.

The TIMMS system may also incorporate surgical workflows. Organized activities such as those observed in the operating room, regardless of complexity, may be better understood and characterized through the process of workflow analysis and diagramming. By analyzing, synthesizing and filtering multi-component processes into their fundamental functional components, a workflow diagram may be generated. To provide consistency and reproducibility this process must utilize a uniform and consistent ontology. The workflow diagram thus generated may be viewed at different levels of granularity or orders. These may be described from the broadest categories (first-order processes) through the finest levels of the surgical procedure (n-order process).

The specific workflow diagrams generated through precise and analytic description of actual surgical procedures may be further distilled into generic, or reference, workflow diagrams for categories of procedures. The reference workflow diagrams thus generated provide the underlying roadmap to be followed by TIMMS throughout an entire operative procedure. This includes each of the three first-order processes: pre-operative assessment and planning; operative procedure; and post-operative care.

The reference workflow diagram is a dynamic and flexible structure, designed to be transformed into a patient-specific workflow, or executing workflow, by TIMMS throughout the entire procedure. The workflow kernel and the various cognitive agents of TIMMS generate a patient-specific model from all of the available sources of data, such as imaging, physiological monitoring, EMR, data repositories, generated simulations, input and feedback from mechatronic devices. Furthermore, on the basis of changes in the patient model throughout the entire procedure, the executing workflow may be modified and updated as necessary. This provides the necessary flexibility required for a surgical procedure in which both minor and major variations are the norm. As variations or deviations from the active executing workflow are encountered, the patient-model and the executing workflow are updated as required. It should be noted that the patient-specific model, available to a physician in real time such as for example, during surgery may be influenced by any and all factors directly impacting the procedure. These include factors that are both intrinsic and extrinsic to the patient, including the functions and status of surgical tools and devices, and activities of the operating surgeon and assistants which may be instantly available to the physician via out devices in an operating room.

As a surgical procedure progresses through the executing workflow, active links between the Workflow Engine and the TIMMS cognitive agents are activated in sequence in order to accomplish the tasks required for TIMMS to help facilitate the surgical process.

In addition, the use of this system will allow the benefits of image guided surgery to be realized for larger populations, as well as individual patients. As more and more cases are performed the results are recorded and analyzed, within the validation engine. This allows outcome analysis and patient safety issues to be evaluated. The system contains the functionality to achieve refinements in medical and surgical “best practices” with reductions in medical errors and enhancements of quality improvement programs. Prospective medical research projects will be more easily achieved through the automated collection, monitoring and measuring of large volumes of data, with numerous variables. Society may begin to reap the benefits of image guided surgery in terms of better outcomes, reduced medical errors, reduced overall cost and liability.

The TIMMS Medical Workstation and the TIMMS Surgical Workstation contain TIMMS software which provides interaction with, access to, and control of the TIMMS engines and TIMMS repositories.

Certain TIMMS software functions may be accessed by the surgeon via a graphical user interface at the TIMMS Medical Workstation and the TIMMS Surgical Workstation. On the other hand, certain TIMMS functions may run automatically in the background.

The overall TIMMS architecture is SOA based and is a reference meta architecture which when implemented consists of a collection of components belonging to four categories. The component categories are: interaction, process, functional components and repositories. Engines, agents and data modules which are part of the components are designed with a granulation level adapted to the application services of a surgical system. The computer software designer, engineer and architect of ordinary skill in the art will know of and understand these concepts, and, with appropriate funding can accomplish the stated purposes and goals of the TIMMS software architecture.

TIMMS is the backbone of a Service Oriented Architecture (SOA) for surgical assistance and may itself consist of one or more engines, agents and data modules. In particular, and according to the various combinations and permutations of different embodiments of the present invention, TIMMS may support one or more of the following process and/or function (not exclusively or in order of priority): 1) allow for flexible and dynamic connection of all service components by realising one or more messaging concepts and data-exchange protocols, e.g. Message Oriented Middleware (MOM) or Service Oriented Architecture Protocol (SOAP); 2) manage the SOA infrastructure to allow for scalable data transmission, for example to fulfil real-time requirements; 3) enable a flexible combination of service components by means of adaptation to non-compatible protocols, data formats and interaction patterns; 4) assist the process component in the orchestration of one or more service components to realise an application domain (e.g. surgical planning or surgical intervention); 5) support a scalable service installation, administration and maintenance, e.g. registration and administration of SOA components; 6) provide operational data service for service documentation and auditing enable web based operations i.e. importing and definition of service in Web Service Definition Language (WSDL) through compatibility with web services architecture; 7) provide transformation of data formats, intelligent routing, message adapting, etc. i.e. include Enterprise Service Bus (ESB) functionalities; 8) support existing DICOM standards relating to diagnostic and therapeutic image management; 9) support future Surgical DICOM extensions of Information Object Definitions (IODs) and Service-Object Pair classes (SOPs); 9) provide policy management facilities; 10) support a selected set of IT standards relating to OR systems.

TIMMS may comprise one or more engines. The Imaging and Biosensors Engine (210A) regulates the connections and flow of images and physiological data. Sources include Repositories, PACS, Electronic Medical record, real-time imaging, real-time patient monitoring, and other sources.

The Modeling Engine regulates the creation and maintenance of the Patient-Model throughout the pre-operative, operative and post-operative stages. It may include a “Patient Model Integrator” which serves as the Cognitive Agent for these processes by enabling a synthesis of different classes of modules. The classes of modules supported may include (not exclusive or in order of priority: 1) geometric modeling including volume and surface representations; 2) properties of cells and tissue; 3) segmentation and reconstruction; 4) biomechanics and damage; 5) tissue growth; 6) tissue shift; 7) prosthesis modelling; 8) fabrication model for custom prosthesis; 9) Properties of biomaterials; 10) atlas-based anatomic modelling; 11) template modelling; 12) FEM of medical devices and anatomic tissue; 13) collision response strategies for constraint deformable objects; 14) variety of virtual human models; 15) lifelike physiology and anatomy; 16) modeling of the biologic continuum; 17) animated models; 18) multi-scale modelling; 19) fusion/integration of data/images; 20) registration between different models incl. patient, equipment; and, 21) modeling of workflows.

The simulation engine provides an environment where surgical procedures can be simulated with an appropriate degree of visual and haptic fidelity as well as timing conditions. The simulation processes may support surgical training as well as pre- and intra-operative planning. It may be based on one or more classes of models provided by the modeling engine. Modeling software having a high degree of sophistication is available from a number of sources and is well within the skill level of one of ordinary skill in the art.

The component of TIMMS called “Kernel for workflow and knowledge and decision management” supports the governance process of a SOA for surgical assist systems. It may consist of one or more process and functional components selected from the group comprising: 1) initiate, support and maintain interventional/surgical processes; 2) control surgical workflows according to rules/guidelines provided by a given healthcare enterprise by executing synchronously or asynchronously one or more function components; 3) operate fully automated or with human interaction; 4) use service from functional components and/or repositories; 5) be driven by (i.e. execute) a given surgical workflow; 6) provide cognitive assistance/agents; 7) build, use and maintain a “Workflow and Knowledge and Decision Tool” repository; 8) the Adaptive Workflow Agent is the Cognitive Agent to evaluate, select, and modify workflows throughout the procedure. The Reference Workflow is selected from the Workflow Repository, based on criteria delineated by the Procedure Class and Code, and by specific information delineated in the Patient-Model.

The Representation and Visualization Engine, engine enables information representation and visualization of n-dimensional data. It also allows interaction with these presentations.

The intervention engine is a component which supports all processes which require human, automatic or semi-automatic interaction with the body of the patient. It includes the management of interventional instruments/devices, navigated control systems, monitoring devices, prosthesis objects, etc. When this engine is executing, it typically has a close link to engines 210A, 210D, 210G.

The validation component may support one or more of the processes selected from the group comprising: 1) assess the surgical workflow activities, in particular the imaging, model and representations accuracy of the surgical intervention; 2) assess specific surgical domain data, information, knowledge and decision presentations, intervention protocols; 3) ascertain that the specific surgical workflow selected fulfils the purpose for which it is intended and is properly executed; 4) ascertain that selected critical activities, which imply given accuracy, precision, real-time response, etc. are properly carried out; 5) ascertain that the appropriate tool sets selected from the repositories will provide the capabilities required; 6) secure that completeness and consistency checks produce the correct results; 7) ascertain that appropriate documentation and reporting for the intervention is carried out; 8) ascertain that the appropriate hardware and software devices required are on-line and functioning

According to the present invention engines hold their associated data objects and algorithmic tool sets and related devices in repositories. Examples of repositories which can be used in association with the engines of the present invention include, but are not limited to, one or more of the following: 1) Images and signals Repository (215A) for the Imaging and Biosensors engine; 2) Modeling tools Repository (215B) for the Modeling engine; 3) Computing tools Repository (215C) for the Simulation engine; 4) Workflow and Knowledge and Decision tools Repository (215D) for the Kernel for Workflow and Knowledge and Decision Management engine; 5) Representation tools Repository (215E) for the Visualization and Representation engine; 6) Devices and Mechatronic tools Repository (215F) for the Intervention engine; 7) Validation tools Repository (215G) for the Validation engine.

One or more additional repositories can also be used within the TIMMS system. These include, for example, a Repository for Models (Simulated Objects) (220). This repository contains generic and patient specific n-dimensional models, associated data sets and intervention plans.

A Repository for Workflow Models, Evidence-Based Medical Data, Case-Based Medical Data (225) which is a repository containing reference and patient specific data sets on pathologies and interventional procedures.

Information and Communication Technology Infrastructure (230) is composed of a distributed network (110) allowing for intercommunication and interactivity between all components of the TIMMS. The Information/Communication Technology infrastructure used by TIMMS includes, structures, objects, processes and interfaces from well established standardized sources, to ensure compatibility. This includes, but is not limited to: IHE (Integrating the Healthcare Enterprise); HIS (Hospital Information System); RIS (Radiology Information System); PACS (Picture Archiving and Communication System); DICOM (Digital Imaging and Communications in Medicine); HL7 (Health Level 7).

External Data and Information Sources (235) includes (but not limited to) links to HIS, RIS and PACS as well information sources from a variety of data bases (atlases, Peer-to-Peer repositories, data grids, etc.) and devices.

Models and Intervention Records (240) including Interventional results and TIMMS generated models are made available for external use through this interface.

The TIMMS system may also include Security and Safety Features. Software and hardware components are included to ensure security and safety features required by regulatory statutes (e.g. HIPAA compliance). This may include, and is not limited to: password protection; varying levels of access by end-users; means of protecting patient confidentiality, error recovery and fault tolerance.

The TIMMS system is extremely flexible and may be operated with varying degrees of automation and user input, depending upon the specific application for which it is being used. The following is a description, in general terms, for TIMMS operation of one embodiment of the present invention. The skilled artisan will appreciate that many variations and modifications are possible are possible, and this description should not be considered limiting in any way.

A TIMMS project is designed to function throughout a surgical workflow at all levels of granularity of each of the three first-order processes: pre-operative assessment and planning; operative procedure; and post-operative care. The initiation of a TIMMS project, in one of many clinical settings, may be considered to take place at the time a request for a procedure is received by the surgeon, and concludes when all post-operative care issues and post-operative quality assurance and archiving activities have been addressed.

This example of a workflow would begin with a Pre-Operative Assessment. When a request for a procedure is received the surgeon may launch the TIMMS software to initialize a new project, at a TIMMS Medical Workstation. (125) The TIMMS Engines (210A-G) and TIMMS Repositories (215A-G, 220, 225) will start up and undergo an automated system check, and all of the engine activities which operate in the background will commence. At this time the Validation Engine (210G) will check that all TIMMS software components are on-line and functioning properly.

The Default Settings of all connected hardware and software devices will be initialized and their proper function will be confirmed by the Validation Engine (210G). At this time the surgeon may modify the specific connections through the TIMMS computer interface.

The workflow would then proceed to Patient input and Procedure Designation step. The surgeon will then establish a new “TIMMS PROJECT” which will have its own unique TIMMS Project ID Number and will enter patient's name and medical record number. In order for TIMMS to begin to select the appropriate Reference Workflow from the Workflow Repository (225) and to perform the data mining from data sources, including the Enterprise-Wide Electronic Medical Record (120, 235, 240), the Surgeon will enter identifying features of the surgical procedure to be performed, such as a Procedure Class and Code. The Reference Workflow would be selected by a cognitive agent of the Kernel for Workflow and Knowledge and Decision Management (Workflow Kernel) (210D). The data mining functions are mediated by the Electronic Medical Record (EMR) Agent of the Workflow Kernel (210D). Patient information and images would be retrieved from the Enterprise-Wide Electronic Medical Record and data sources (120, 235, 240) including the Radiology Information System (RIS), Hospital Information System (HIS), PACS (Picture Archiving and Communications System).

A TIMMS cognitive agent, the EMR Agent, performs retrieval of data from the Enterprise-Wide Electronic Medical record and the data sources. (120, 235, 240) This includes all relevant patient information, such as history and physical, past medical history, laboratory data, pathology reports, consultations, etc. The Imaging Agent of the Imaging and Biosensors Engine (210A) will also retrieve and download pertinent medical imaging studies.

Once the required patient information and images are retrieved and made available, the next step is determining whether or not the proposed procedure is appropriate and indicated.

One of the core functions throughout the TIMMS project is the creation and maintenance of the patient-model. A cognitive agent of the TIMMS Modeling Engine ((210B), the Patient-Model Integrator, which creates and updates the patient-model, will be activated. The information compiled in the patient-model is used to determine whether the patient is a suitable candidate for undergoing the proposed treatment and if the features of the underlying pathology are favorable for this treatment. Examples of parameters collected and analyzed would include the features of a tumor (histological characteristics; stage, grade, size/volume; shape; proximity to skin, organs, vessels; blood vessel patency and flow; imaging features (CT, Ultrasound, MRI, PET characteristics); and, previous treatment (such as, systemic chemotherapy, surgery, chemoembolization). Information obtained from the previously retrieved images would be used to determine feasibility of treatment based on anatomical features.

Another of the core functions of a TIMMS project according to this exemplary embodiment of the present invention is the selection of the Reference Workflow and its modification into an Executing Workflow which is updated as changes in the patient-model are encountered. The Adaptive Workflow Agent and the Treatment Assessment Simulator of the Workflow Kernel (210D) and the Simulation Engine (210C), respectively, would be instrumental in determining suitability of the proposed treatment and in selecting the appropriate Reference Workflow. A group of possible Reference Workflows would be selected, simulations would be conduct, and the “best-fit” Reference Workflow would be selected. The Reference Workflow would then be transformed into the Executing Workflow based on the specific features delineated in the patient-model. This Executing Workflow forms the basis for the treatment plan.

Cognitive agents of the Workflow Kernel (210D) and the Validation Engine (210G), such as the Outcomes Predictor, then perform data mining and outcomes predictions. The patient-model, the Executing Workflow, and data mined from Enterprise-Wide Electronic Medical record and the data sources (120, 235, 240), are analyzed by the Surgeon, assisted by the Workflow Kernel (210D), to provide a prospective quantitative and qualitative assessment of the likelihood of technical success.

When the Surgeon determines that the patient is a suitable candidate, the Scheduling Agent of the Workflow Kernel (210D) will proceed to schedule the procedure, and the TIMMS will continue to update the patient-model and Executing Workflow in the background, as any additional information, such as laboratory data collected during pre-surgical testing, is accumulated.

If needed, 3-D illustrations or 3-D models to facilitate surgery are created by the Representation and Visualization Engine (210E) and Modeling Engine (210B). Through its Information and Communication Technology Infrastructure (230), TIMMS is capable of remotely initiating the design and building of surgical 3-D models by Model Building Devices (140D) that are networked to TIMMS.

The next step would be the Operative Procedure with a possible first substep such as a Refinement of Workflow. On the day of the operative procedure, after the TIMMS is started up and its functions and connections are checked and the physiological monitoring has been initiated, the Patient Model Integrator updates patient-model from real-time physiologic data. Revisions to Workflow are suggested by the Adaptive Workflow Agent of the Workflow Kernel (210D) as the Patient Model Integrator updates the patient-model.

Prior to the onset of the administration of anesthesia and the onset of the surgical procedure, pre-anesthesia assessment is required to ensure patient safety. Patient data is acquired from Monitoring and Sensor Devices (140C) by the efforts of the Imaging and Biosensors Engine (210A) and the Patient Safety Agent of the Validation Engine (210G), and/or entered by operating room personnel. The procedure can only commence when the pre-anesthesia assessment is complete.

At the onset of the procedure the cognitive agents of the Workflow Kernel (210D) monitor the procedure in parallel with the evolving Executing Workflow, recording the actual Executing Workflow ultimately used.

Mechatronic and Other Surgical Devices (140A) and Navigation Devices (140F) will now come into play in this example. As the procedure progresses, the flow of images and data through the Information and Communication Technology Infrastructure (230) is maintained between the imaging Devices (140E) (e.g. the CT scanner and/or ultrasound) and the Registration and Navigation Agents of the Intervention Engine (210F). Mechatronic and other Surgical Devices (140A), Positioning Devices (140B), Navigation Devices 140F) are brought on-line. All available imaging and physiologic data is fed through the Information and Communication Technology Infrastructure (230) to the Mechatronic and other Surgical Devices (140A), Positioning Devices (140B), Navigation Devices 140F) for maximum operative precision. This data is also assimilated by the Adaptive Workflow Agent into the Executing Workflow.

Any additional Monitors and Visualization Devices (155), such as stereoscopic overlay, are brought on-line, with all available data and images input from the Representation and Visualization Engine (210E).

Once all available data has been processed by TIMMS, the Adaptive Workflow Agent makes final revisions to the Executing Workflow, and the efficacy of the proposed treatment is confirmed through the Validation Engine (210G).

When the final, specified plan is completed and displayed, the Surgeon then proceeds with skin preparation and administration of anesthesia. The Operation commences according to the Executing Workflow.

The Biosensor and Imaging Engine (210A), Intervention Engine (210F) and Validation Engine (210G) enable coordinated, synchronized function of real-time Imaging Devices (140E)(such as CT, Fluoroscopy and/or ultrasound); registration software, along with any navigation and mechatronic devices.

The procedure proceeds with close monitoring of the Executing Workflow, with feedback from the surgeon, biosensors, and monitors. Intra-operative images and data from biosensors will be analyzed and the patient model will be updated through a synchronized effort of the Imaging and Biosensors Engine (210A), the Modeling Engine(210B), the Workflow Kernel (210D), and the Validation engine (210G). If necessary, the Adaptive Workflow Agent will suggest changes to the Executing Workflow based on current data, for any suggested modifications to the treatment plan. Feedback from Navigational Devices (140F) will also dictate modifications to the Executing Workflow by the Adaptive Workflow Agent.

The TIMMS system may then extend into post-operative care. After the procedure is completed, the patient may be continuously monitored with data streams from any data source including lab test, new images, heart monitor, etc. with the Imaging and Biosensors Engine (210A) updating the patient model.

After the patient outcome is determined according to common medical procedures the TIMMS system can begin the Validation Process. For example, the EMR Agent will update the patient's medical records with a report of the procedure and its outcome. The Validation Engine (210G) will perform a variety of validation functions, including outcomes analysis, statistical evaluation, complication recording, etc. This data is sent to Repository for Workflows, Evidence-Based Medicine, and Cases (225) and the Enterprise-Wide Electronic Medical Record (120), and will be available for additional evaluation and research purposes. All required Quality Assurance procedures and documentation will be completed.

When post-operative assessment indicates that patient is stable and ready for transfer, and when Validation procedures have been completed, the Patient Safety Agent of the Validation Engine (210G) can indicate that the patient is ready for transfer to the Recovery Room.

While this is an example of one embodiment of the present invention, described in terms of Image Guided Surgery, the process described herein will find application in any and all fields of complex human endeavor, where large volumes of data, from a plurality of sources, need to be integrated, in real-time, to help ensure accuracy, safety and guidance. Therefore, this invention will apply to other health care services, as well as other industries.

The present invention has been described in terms of specific embodiments, and it is recognized that equivalents, alternatives, and modifications, aside from those expressly stated, are possible and within the scope of the appended claims.

I (we) claim:

1. A method of performing surgery on a patient comprising the step of comparing a chosen patient's data to statistical data in a repository of patient data to develop a patient specific model, wherein the data comprises information from two or more sub databases selected from the group consisting of workflow data, electronic medical records, diagnostic data, biological data, measurement data, anatomical data, physiological data, pathological data, genetic data, molecular data, imaging data, chemical data, clinical laboratory data, simulated data, coordinate data and surgical result and wherein the patient specific model aids in the preoperative, operative or post operative phase of surgery performed in real time on the patient.

2. A method of performing surgery according to claim 1 wherein the chosen patient's data comprises information from three or more sub databases selected from the group consisting of workflow data, electronic medical records, diagnostic data, anatomical data, physiological data, pathological data, genetic data, molecular data, imaging data, chemical data, clinical laboratory data, biological data, simulated data, measurement data, coordinate data and surgical result.

3. A method of performing surgery according to claim 1 wherein the chosen patient's data comprises information from four or more sub databases selected from the group consisting of workflow data, electronic medical records, diagnostic data, anatomical data, physiological data, pathological data, genetic data, molecular data, imaging data, chemical data, clinical laboratory data, biological data, simulated data, measurement data, coordinate data and surgical result.

4. A method of performing surgery according to claim 1 wherein the chosen patient's data comprises information from five or more sub databases selected from the group consisting of workflow data, electronic medical records, diagnostic data, anatomical data, physiological data, pathological data, genetic data, molecular data, imaging data, chemical data, clinical laboratory data, biological data, simulated data, measurement data, coordinate data and surgical result.

5. A method of performing surgery according to claim 1 wherein the chosen patient's data comprises information from six or more sub databases selected from the group consisting of workflow data, electronic medical records, diagnostic data, anatomical data, physiological data, pathological data, genetic data, molecular data, imaging data, chemical data, clinical laboratory data, biological data, simulated data, measurement data, coordinate data and surgical result.

6. A method of performing surgery according to claim 1 wherein the method is performed in real time before surgery.

7. A method of performing surgery according to claim 1 wherein the method is performed in real time during surgery.

8. A method of performing surgery according to claim 1 wherein the method is performed in real time after a patient has undergone surgery.

9. A method according to claim 1 wherein the patient specific model data is deposited into the statistical data in a repository of patient data.

10. A method according to claim 1 wherein the statistical data in a repository of patient data is connected to databases in other interested organizations.

11. A repository, communications and computer service oriented architecture for surgical assistance comprising:
a first element consisting of two or more electronic repositories selected from the group consisting of workflow data, electronic medical records, diagnostic data, anatomical data, physiological data, pathological data, genetic data, molecular data, imaging data, chemical data, clinical laboratory data, biological data, simulated data, measurement data, coordinate data and surgical result;
a second element consisting of a means for communications between medical personnel, the two or more electronic repositories and one or more engines making up a third element; and,
a third element consisting of one or more engines which generate, analyze, evaluate, or manage input and output; and wherein,
the three elements are connected to each other such that patient specific data input or manipulated by a medical professional can be compared to statistical data or generic models, said statistical data or generic models derived from previously entered patient specific data.

12. A repository, communications and computer service oriented architecture according to claim 11 wherein a part or all of the first element is located at a remote location.

13. A repository, communications and computer service oriented architecture according to claim 11 wherein the one or two of the first, second or third elements are owned by a different party than the owner of the other elements.

14. A repository, communications and computer service oriented architecture according to claim 11 wherein the engine is a special purpose software module.

15. A method of performing surgery comprising the steps of:
a) preparing a patient specific model for a first patient comprising two or more fields from the group consisting of workflow data, electronic medical records, diagnostic data, anatomical data, physiological data, pathological data, genetic data, molecular data, imaging data, chemical data, clinical laboratory data, biological data, simulated data, measurement data, coordinate data and surgical result;
b) comparing said patient specific data from said first patient to statistical data or generic models based upon information from a statistically significant number of patients;
c) employing said comparisons in a useful manner before, during or after surgery.

16. An electronic patient specific model comprising: a computer based data set comprising a first set of information specific to one patient, a second set of information from a statistical sampling of other patents having similar symptoms or diagnosis and a third set of information comprising an analysis of the first two sets of information and wherein the third set of information is useful in the diagnosis or treatment of a patient.

17. An electronic patient specific model according to claim 16 wherein the third set of information comprises a comparison of mean, average or standard deviations between the first set of information and the second set of information.

18. An electronic Patient specific model according to claim 16 wherein the third set of data may also comprise workflow data regarding possible diagnosis, treatment or complication options.

19. An electronic patient specific model according to claim 16 wherein the third set of data may also comprise outcome data.

20. An electronic patient specific model according to claim 16 wherein the third set of data may also comprise billing data.

21. An electronic patient specific model according to claim 16 wherein the third set of data may also comprise electronic data.

22. An electronic patient specific model according to claim 16 wherein the third set of data may also comprise an atlas based anatomical model.

23. An electronic patient specific model according to claim 16 wherein the third set of data may also comprise an image based anatomical model with coordinate data regarding a part or whole of a patient's body.
